(19) United States
(12) Patent Application Publication
(10) Pub. No.: US 2008/0194632 A1
(43) Pub. Date: Aug. 14, 2008

(54) Novel Piperidine Derivatives as Chemokine Receptor Modulators Useful for the Treatment of Respiratory Diseases

(75) Inventors: Julien Giovannini, Leicestershire (GB); Bo-Goran Josefsson, Lund (SE); Marguerite Mensonides-Harsema, Lund (SE); Hakan Schulz, Lund (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/997,474

(22) PCT Filed: Jul. 31, 2006
(86) PCT No.: PCT/SE2006/000918

§ 371 Date: Jan. 31, 2008

(30) Foreign Application Priority Data
Aug. 1, 2005 (SE) 0501757-9

(51) Int. Cl. A61K 31/4468; C07D 211/56; A61P 11/00; A61P 29/00

(52) U.S. Cl. 514/329; 546/224

(57) ABSTRACT

The invention provides compounds of formula wherein m, R¹, R² and R³ are as defined in the specification, salts and polymorphic forms thereof, processes for the preparation of the compounds, salts and polymorphs, pharmaceutical compositions containing these compounds, salts and/or polymorphic forms and their use in therapy.

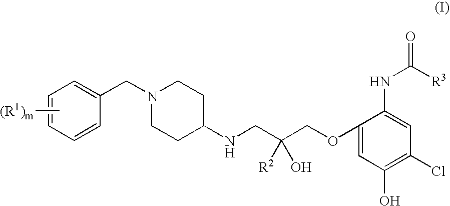

The present invention relates to novel compounds, salts and polymorphic forms thereof, processes for the preparation of the compounds, salts and polymorphs, pharmaceutical compositions containing these compounds, salts or polymorphic forms and their use in therapy.

Chemokines play an important role in immune and inflammatory responses in various diseases and disorders, including asthma, allergic diseases, rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C) and Cys-Cys (C—C) families. These are distinguished on the basis of a single amino acid insertion between the NH-proximal pair of cysteine residues and sequence similarity.

Chemokines are attractants and activators of monocytes, lymphocytes and neutrophils. The C—C chemokines include potent chemoattractants such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β). The C—X—C chemokines include several potent chemoattractants such as interleukin-8 (IL-8) and neutrophil-activating peptide 2 (NAP-2). Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXC2, CXCR3 and CXCR4. These receptors represent good targets for drug development since agents which modulate these receptors would be useful in the treatment of disorders and diseases such as those mentioned above.

International publication numbers WO01/98273 and WO03/051839 describe benzyl-piperidines which modulate MIP-1α chemokine receptor activity.

A desirable property for a drug acting at the CCR1 receptor is that it has high potency e.g. as determined by its ability to inhibit the activity of the CCR1 receptor. It is also desirable for such drugs to possess good selectivity and pharmacokinetic properties in order to further enhance drug efficacy. As an example, it can be advantageous for such drugs to possess good metabolic stability.

It is also desirable for compounds to exhibit low activity against the human ether-a-go-go-related-gene (hERG)-encoded potassium channel. In this regard, low activity against hERG binding in vitro is indicative of low activity in vivo.

The present inventors have identified new compounds which modulate CCR1 receptor activity and which have particularly beneficial potency, selectivity and/or pharmacokinetic properties.

Chemical stability, solid state stability, and “shelf life” of the active ingredients are important factors. The drug substance, and compositions containing it, should preferably be capable of being effectively stored over appreciable periods of time, without exhibiting a significant change in the active component's physico-chemical characteristics (e.g. its chemical composition, density, hygroscopicity and solubility). Moreover, it is also important to be able to provide drugs in a form, which is as chemically pure as possible. The skilled person will appreciate that, typically, if a drug can be readily obtained in a stable form, such as a stable crystalline form, advantages may be provided, in terms of ease of handling, ease of preparation of suitable pharmaceutical compositions, and a more reliable solubility profile.

### DESCRIPTION OF THE DRAWINGS

FIG. 1. X-ray powder diffraction peaks of N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph A).

FIG. 2. X-ray powder diffraction peaks of N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph B).

FIG. 3. X-ray powder diffraction peaks of N-{5-Chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph A)

FIG. 3. X-ray powder diffraction peaks of N-{5-Chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph B).

In accordance with the present invention, there is provided a compound of formula

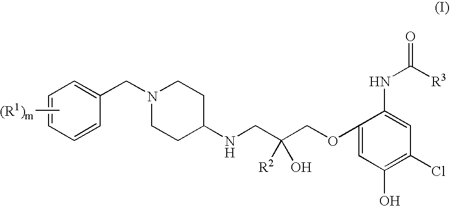

wherein

m is 1 or 2;

each R¹ independently represents halogen;

R² represents a hydrogen atom or a methyl group; and

R³ represents C₁-C₄ alkyl;

or a pharmaceutically acceptable salt or solvate thereof.

The present inventors have, inter alia, surprisingly found that a particular substitution pattern on the right hand side of the structure shown in formula (I) gives rise to an enhancement in potency against CCR1 receptor activity. Without being bound to any particular theory, it is believed that this advantageous property is at least due in part to the presence of, and point of attachment of, the substituents on the benzene ring on the right-hand side of the molecule in formula (I).

In the context of the present specification, an alkyl substituent group may be linear or branched.

R¹ represents a halogen atom such as a fluorine, chlorine, bromine or iodine atom, particularly a chlorine atom.

In one embodiment of the invention, m is 1 and R¹ represents a halogen atom, particularly a chlorine atom.

In a further embodiment, m is 1 and R¹ represents a halogen atom (e.g. chlorine) in the 4-position of the benzene ring relative to the carbon atom to which the CH₂ linking group is attached.

In another embodiment of the invention, R² represents a methyl.

R³ represents C₁-C₄ alkyl (e.g. methyl, ethyl, n-propyl, isopropyl, n-butyl, isobutyl, tert-butyl).

Typically, R³ is methyl or ethyl.

In another embodiment of the invention, m is 1 and R¹ represents a halogen atom in the 4-position of the benzene ring relative to the carbon atom to which the CH₂ linking group is attached, R² represents hydrogen or a methyl group and R³ represents C₁-C₄ alkyl.

In a further embodiment of the invention, m is 1 and R¹ represents a halogen atom in the 4-position of the benzene ring relative to the carbon atom to which the CH₂ linking group is attached, R² represents hydrogen or a methyl group and R³ represents methyl or ethyl.

In a further embodiment of the invention, m is 1 and R¹ represents a chlorine atom in the 4-position of the benzene ring relative to the carbon atom to which the CH₂ linking group is attached, R² represents a methyl group and R³ represents methyl or ethyl.

In another embodiment, the compounds of the invention include the following compounds or a pharmaceutically acceptable salt or solvate thereof:
N-{5-Chloro-2-[((2S)-3-{[1-(4-chlorobenzyl)piperidin-4-yl]amino}-2-hydroxypropyl)oxy]-4-hydroxyphenyl}acetamide,
N-{5-Chloro-2-[((2S)-3-{[1-(4-chlorobenzyl)piperidin-4-yl]amino}-2-hydroxy-2-methylpropyl)oxy]-4-hydroxyphenyl}acetamide,
N-{5-Chloro-2-[((2S)-3-{[1-(4-chlorobenzyl)piperidin-4-yl]amino}-2-hydroxypropyl)oxy]-4-hydroxyphenyl}propaneamide, or
N-{5-Chloro-2-[((2S)-3-{[1-(4-chlorobenzyl)piperidin-4-yl]amino}-2-hydroxy-2-methylpropyl)oxy]-4-hydroxyphenyl}propaneamide.

The present invention further provides a process for the preparation of a compound of formula (I) or a pharmaceutically acceptable salt or solvate thereof as defined above which comprises

(a) reacting a compound of formula

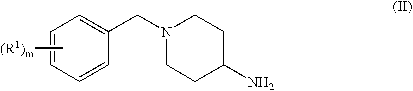

wherein m and R¹ are as defined in formula (I), with a compound of formula

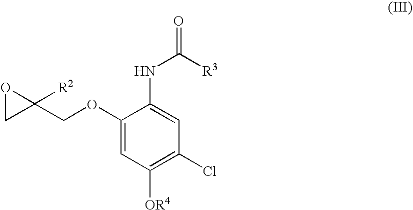

wherein R² and R³ are as defined in formula (I), and R⁴ represents a hydrogen atom or a suitable protecting group; or
(b) reacting a compound of formula

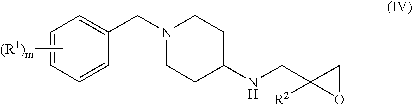

wherein m, R¹ and R² are as defined in formula (I), with a compound of formula

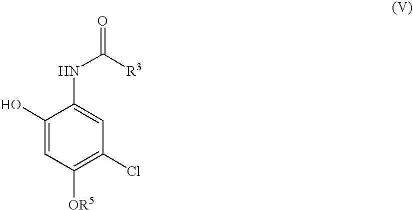

wherein R³ is as defined in formula (I), and R⁵ represents a hydrogen atom or a suitable protecting group; or
(c) reacting a compound of formula

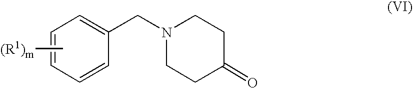

wherein m and R¹ are as defined in formula (I), with a compound of formula

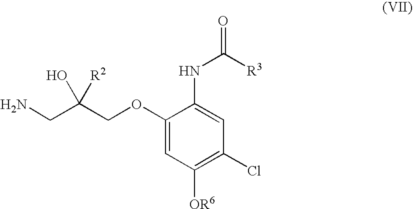

wherein R² and R³ are as defined in formula (I), and R⁶ represents a hydrogen atom or a suitable protecting group;
and optionally after (a), (b) or (c) forming a pharmaceutically acceptable salt or solvate of the compound of formula (I).

The process of the invention may conveniently be carried out in a solvent, e.g. an organic solvent such as an alcohol (e.g. methanol or ethanol), a hydrocarbon (e.g. toluene), THF or acetonitrile at a temperature of, for example, 15° C. or above such as a temperature in the range from 20 to 120° C.

Compounds of formulae (II), (III), (IV), (V), (VI) and (VII) are either commercially available, are well known in the literature or may be prepared easily using known techniques.

It will be appreciated by those skilled in the art that in the process of the present invention certain functional groups such as hydroxyl or amino groups in the starting reagents or intermediate compounds may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

Compounds of formula (I) above may be converted to a pharmaceutically acceptable salt or solvate thereof, preferably an acid addition salt such as a hydrochloride, hydrobromide, phosphate, sulphate, acetate, ascorbate, benzoate, fumarate, hemifumarate, furoate, succinate, maleate, tartrate, citrate, oxalate, xinafoate, methanesulphonate or p-toluenesulphonate. A pharmaceutically acceptable salt also includes internal salt (zwitterionic) forms.

One embodiment of the invention relates to the benzoate and furoate salts of the compounds of formula I. Another embodiment relates to N-{5-chloro-2-[((2S)-3-{[1-(4-chlorobenzyl)piperidin-4-yl]amino}-2-hydroxy-2-methylpropyl)oxy]-4-hydroxyphenyl}acetamide benzoate. A further embodiment relates to N-{5-chloro-2-[((2S)-3-{[1-(4-chlorobenzyl)piperidin-4-yl]amino}-2-hydroxy-2-methylpropyl)oxy]-4-hydroxyphenyl}acetamide furoate.

The compounds of formula (I) are capable of existing in stereoisomeric forms. It will be understood that the invention encompasses the use of all geometric and optical isomers of the compounds of formula (I) and mixtures thereof including racemates. The use of tautomers and mixtures thereof also form an aspect of the present invention. Preferred optical isomers are the (S)-enantiomers (i.e. compounds with the S configuration at the stereocentre with R² and OH attached).

One embodiment of the invention relates to N-{5-chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph A), which exhibits at least the following characteristic X-ray powder diffraction (XRPD) peaks at d-values at;

6.0, 16.3 and 19.1 or
6.0, 9.5, 15.4, 16.3 and 19.1 or
6.0, 14.9, 19.1 and 24.2 or
6.0, 9.5, 12.0, 15.4, 16.3 and 24.2 or
6.0, 12.0, 14.9, 15.4, 16.3, 19.1 and 24.2 Å.

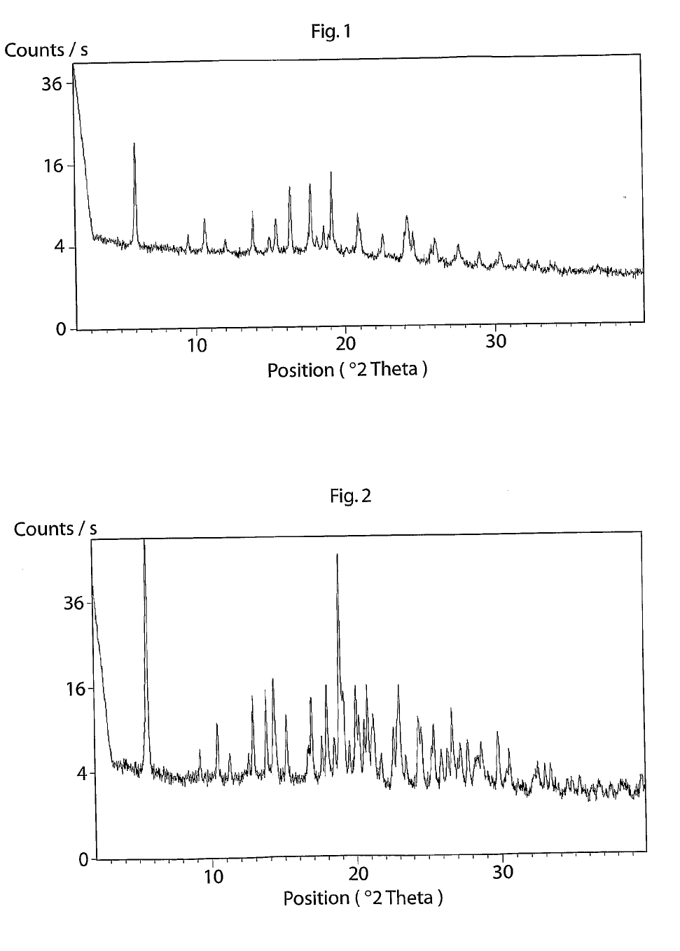

The invention relates to the XRPD peaks as shown in FIG. 1.

Another embodiment of the invention relates to N-{5-chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph B), which exhibits at least the following characteristic X-ray powder diffraction (XRPD) peaks at d-values at;

5.6, 10.4 and 14.3 or
9.2, 12.9, 16.9, 19.5, and 25.4 or
5.6, 9.2, 11.3, 14.3, 16.9, 20.0 and 23.0 or
5.6, 11.3, 12.9, 18.0, 19.5, and 20.0, 23.0, 25.4 or,
5.6, 9.2, 10.4, 12.9, 14.3, 16.9, 18.0, 19.5, 20.0 and 25.4 or
5.6, 9.2, 10.4, 11.3, 12.9, 14.3, 16.9, 18.0, 19.5, 23.0 and 25.4 or
5.6, 9.2, 10.4, 11.3, 14.3, 16.9, 18.0, 19.5, 20.0, 23.0 and 25.4 or
5.6, 9.2, 10.4, 11.3, 12.9, 14.3, 16.9, 18.0, 19.5, 20.0, 23.0 and 25.4 Å.

The invention relates to the XRPD peaks as shown in FIG. 2.

A further embodiment of the invention relates to N-{5-chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph A), which exhibits at least the following characteristic X-ray powder diffraction (XRPD) peaks at d-values at;

6.4, 16.8 and 18.1 or
6.4, 10.8, 15.8, 16.8, 18.1 and 19.2 or
6.4, 9.8, 10.8, 15.5, 15.8, 16.8, 17.4, 18.1 and 25.7 or
6.4, 10.8, 15.5, 15.8, 16.8, 18.1, 19.2, 19.6 and 21.9 or
6.4, 10.8, 15.5, 15.8, 16.8, 18.1, 19.2, 19.6, 21.9 and 25.7
6.4, 9.8, 10.8, 15.5, 15.8, 16.8, 18.1, 19.2, 19.6, 21.9 and 25.7 or
6.4, 9.8, 10.8, 15.5, 15.8, 16.8, 17.4, 18.1, 19.2, 19.6, 21.9 and 25.7 Å.

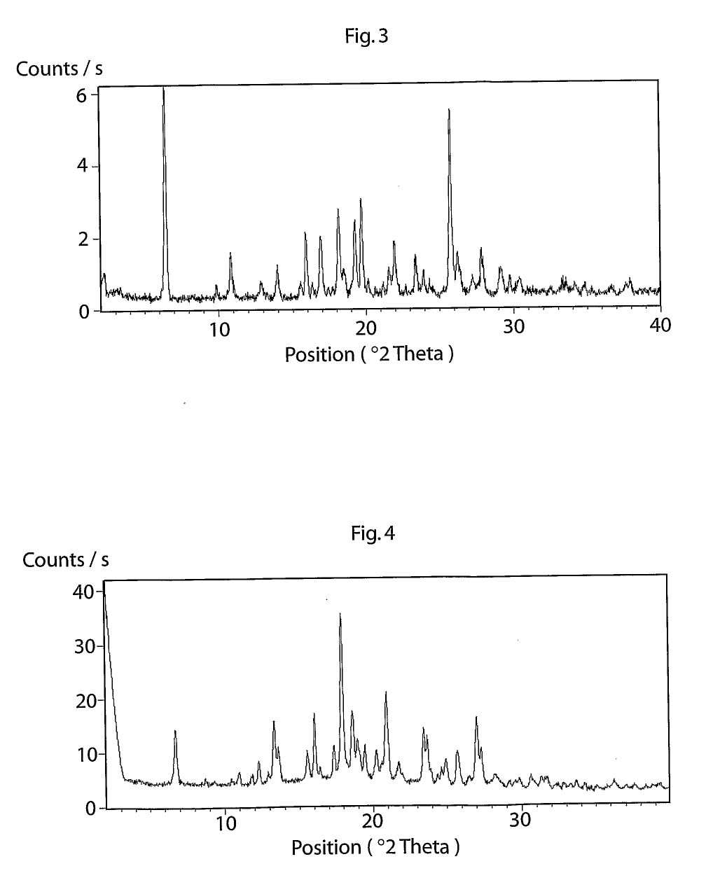

The invention relates to the XRPD peaks as shown in FIG. 3.

Yet another embodiment of the invention relates to N-{5-chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph B), which exhibits at least the following characteristic X-ray powder diffraction (XRPD) peaks at d-values at;

6.7, 17.9 and 20.9 or
12.2, 13.3, 17.9 and 18.6 or
6.7, 12.2, 16.0, 17.3 and 27.0 or
6.7, 8.7, 12.2, 13.3, 16.0, 17.9 and 18.6 or
6.7, 12.2, 13.3, 16.0, 17.3, 17.9, 18.6, 20.9 and 27.0 or
6.7, 12.2, 13.3, 13.6, 15.5, 16.0, 17.3, 17.9, 18.6, 19.4, 20.9 and 27.3 or
6.7, 12.2, 13.3, 13.6, 15.5, 16.0, 17.3, 17.9, 18.6, 19.4, 20.9, 23.4 and 23.6 or
6.7, 12.2, 13.3, 13.6, 15.5, 16.0, 17.3, 17.9, 18.6, 19.4, 20.9, 23.4, 23.6, 27.0 and 27.3 Å.

The invention relates to the XRPD peaks as shown in FIG. 4.

The method for preparing the salt forms may vary. The preparation of N-{5-chloro-2-[((2S)-3-{[1-(4-chlorobenzyl)piperidin-4-yl]amino}-2-hydroxy-2-methylpropyl)oxy]-4-hydroxyphenyl}acetamide salt forms involves (i) the free base or a solution of the free base of suitably pure N-{5-chloro-2-[((2S)-3-{[1-(4-chlorobenzyl)piperidin-4-yl]amino}-2-hydroxy-2-methylpropyl)oxy]-4-hydroxyphenyl}acetamide in a suitable solvent is mixed with any of the acids in pure form or as a solution of acid(s) in a suitable solvent (typically 0.5 to 1 equivalents of the acid),

(iia) cooling the resulting salt solution if necessary to cause precipitation, or
(iib) adding a suitable anti-solvent to cause precipitation, or
(iic) evaporating the first solvent and adding and new solvent and repeating either steps (iia) or step (iib), and
(iii) filtering and collecting the salt.

The stoichiometry, solvent mix, solute concentration and temperature employed may vary. Representative solvents that may be used to prepare and/or recrystallize the salt forms include, without limitation, ethanol, methanol, furoic acid, butanol, isopropyl alcohol, dichloromethane, acetone, ethylacetate, and acetonitrile.

According to a further embodiment of the invention there is provided the benzoate and furoate salt of the invention in substantially crystalline form.

The benzoate and furoate salts of the invention may be produced in forms which are greater than 80% crystalline, by “substantially crystalline” we include greater than 20%, preferably greater than 30%, and more preferably greater than 40% (e.g. greater than any of 50, 60, 70, 80 or 90%) crystalline. One embodiment refers to the benzoate and furoate salts of the invention in forms, which are 70% to 90%, preferably 75% to 85% crystalline. According to a further aspect of the invention there is also provided a benzoate and furoate salt of the invention in partially crystalline form. By “partially crystalline” we include 5% or between 5% and 20% crystalline.

The degree (%) of crystallinity may be determined by the skilled person using X-ray powder diffraction (XRPD). Other techniques, such as solid state NMR, FT-IR, Raman spectroscopy, differential scanning calorimetry (DSC) and microcalorimetry, may also be used.

It will be appreciated that the compounds of formula (I) and salts thereof may exist as zwitterions. Thus, whilst the compounds are drawn and referred to in the hydroxyl form, they may exist also in internal salt (zwitterionic) form. The representation of formula (I) and the examples of the present invention covers both hydroxyl and zwitterionic forms and mixtures thereof in all proportions.

The compounds of formula (I), salts and polymorphs thereof, have activity as pharmaceuticals, and are surprisingly potent modulators of chemokine receptor (especially CCR1 receptor) activity, and may be used in the treatment of autoimmune, inflammatory, proliferative and hyperproliferative diseases and immunologically-mediated diseases.

A compound of the invention, or a pharmaceutically acceptable salt thereof, can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antiitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiform; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinophilic gastro-enteritis, mastocytosis, Crohn's disease, ulcerative colitis, microscopic colitis, indeterminate colitis, irritable bowel disorder, irritable bowel syndrome, non-inflammatory diarrhea, food-related allergies which have effects remote from the gut, e.g., migraine, rhinitis and eczema.

Thus, in a further aspect, the present invention provides a compound of formula (I) or a pharmaceutically-acceptable salt or solvate thereof or polymorphic forms thereof, as hereinbefore defined for use in therapy.

In a further aspect, the present invention provides a method of treating a respiratory disease in a patient suffering from, or at risk of, said disease, which comprises administering to the patient a therapeutically effective amount of a compound of formula (I) or a pharmaceutically acceptable salt or solvate thereof or polymorphic forms thereof, as hereinbefore defined.

In a further aspect, the present invention provides the use of a compound of formula (I) or a pharmaceutically-acceptable salt or solvate thereof or polymorphic forms thereof, as hereinbefore defined in the manufacture of a medicament for use in treating a respiratory disease.

In a further aspect, the present invention provides a method of treating an airways disease in a patient suffering from, or at risk of, said disease, which comprises administering to the patient a therapeutically effective amount of a compound of formula (I) or a pharmaceutically acceptable salt or solvate thereof or polymorphic forms thereof, as hereinbefore defined.

In a further aspect, the present invention provides a method of treating an inflammatory disease in a patient suffering from, or at risk of, said disease, which comprises administering to the patient a therapeutically effective amount of a compound of formula (I), or a pharmaceutically acceptable salt or solvate thereof or polymorphic forms thereof, as hereinbefore defined.

In a further aspect, present invention provides the use of a compound of formula (I) or a pharmaceutically-acceptable salt or solvate thereof or polymorphic forms thereof, as hereinbefore defined in the manufacture of a medicament for use in treating an inflammatory disease.

In a further aspect, the present invention provides the use of a compound of formula (I) or a pharmaceutically-acceptable salt or solvate thereof or polymorphic forms thereof, as hereinbefore defined in the manufacture of a medicament for use in treating an airways disease.

In a further aspect, the present invention provides a method of treatment of respiratory disease and/or asthma, in a patient suffering from, or at risk of, said disease, which comprises administering to the patient a therapeutically effective amount of a compound of formula (I), or a pharmaceutically acceptable salt or solvate thereof or polymorphic forms thereof, as hereinbefore defined.

An agent for the treatment of inflammatory disease, respiratory disease and/or asthma, which comprises as active ingredient a compound of formula (I) or a pharmaceutically-acceptable salt or solvate thereof or polymorphic forms thereof.

In a further aspect, the present invention provides the use of a compound of formula (I) or a pharmaceutically-acceptable salt or solvate thereof or polymorphic forms thereof, as hereinbefore defined in the manufacture of a medicament for use in treating asthma or chronic obstructive pulmonary disease.

In a further aspect, the present invention provides the use of a compound of formula (I) or a pharmaceutically-acceptable salt or solvate thereof or polymorphic forms thereof, as hereinbefore defined in the manufacture of a medicament for the treatment of human diseases or conditions in which modulation of CCR1 activity is beneficial.

In the context of the present specification, the term “therapy” also includes “prophylaxis” unless there are specific indications to the contrary. The terms “therapeutic” and “therapeutically” should be construed accordingly.

For the above-mentioned therapeutic uses the dosage administered will, of course, vary with the compound employed, the mode of administration, the treatment desired and the disorder indicated. The daily dosage of the compound of formula (I) may be in the range from 0.001 mg/kg to 30 mg/kg.

The compound of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvants, diluents and/or carriers. Depending on the mode of administration, the pharmaceutical composition will preferably comprise from 0.05 to 99%/w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore defined, in association with pharmaceutically acceptable adjuvants, diluents and/or carriers.

The invention further provides a process for the preparation of a pharmaceutical composition of the invention, which comprises mixing a compound of formula (I), or a pharmaceutically acceptable salt or solvate thereof, as hereinbefore defined, with a pharmaceutically acceptable adjuvants, diluents and/or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. In a preferred embodiment, the compositions of the invention are administered topically by inhalation.

The term “stability” as defined herein includes chemical stability and solid-state stability. By “chemical stability”, we include that it may be possible to store salts of the invention in an isolated form, or in the form of a formulation in which it is provided in admixture with pharmaceutically acceptable carriers, diluents or adjuvants, under normal storage conditions, with an insignificant degree of chemical degradation or decomposition. By “solid state stability”, we include that it may be possible to store salts of the invention in an isolated solid form, or in the form of a solid formulation in which it is provided in admixture with pharmaceutically acceptable carriers, diluents or adjuvants, under normal storage conditions, with an insignificant degree of solid state transformation (e.g. crystallisation, recrystallisation, solid state phase transition, hydration, dehydration, solvatisation or desolvatisation).

Examples of “normal storage conditions” include temperatures of between minus 80 and plus 50° C. (preferably between 0 and 40° C. and more preferably room temperatures, such as 15 to 30° C.), pressures of between 0.1 and 2 bars (preferably at atmospheric pressure), relative humidity of between 5 and 95% (preferably 10 to 60%), and/or exposure to 460 lux of UV/visible light, for prolonged periods (i.e. greater than or equal to six months). Under such conditions, salts of the invention may be found to be less than 15%, more preferably less than 10%, and especially less than 5%, chemically degraded/decomposed, or solid state transformed, as appropriate. The skilled person will appreciate that the above-mentioned upper and lower limits for temperature, pressure and relative humidity represent extremes of normal storage conditions, and that certain combinations of these extremes will not be experienced during normal storage (e.g. a temperature of 50° C. and a pressure of 0.1 bar).

The invention will now be further explained by reference to the following illustrative examples.

¹H NMR spectra were recorded on a Varian Unity Inova 400 or a Varian Mercury VX 300 and data are quoted in the form of delta values, given in parts per million (ppm) relative to tetramethylsilane (TMS) as an internal standard.

The central solvent peak of chloroform-d (δ_{H} 7.27 ppm), acetone-d₆ (δ_{H} 2.05 ppm), or DMSO-d₆ (δ_{H} 2.50 ppm) were used as internal standard.

Low resolution mass spectra and accurate mass determination were recorded on an Agilent MSD 1100 LC-MS system equipped with APCI/ESI ionisation chambers. All solvents and commercial reagents were laboratory grade and used as received.

X-ray powder diffraction (XRPD) analyses were performed on samples prepared according to standard methods (see for example Giacovazzo et al., eds., Fundamentals of Crystallography, Oxford University Press (1992); Jenkins & Snyder, eds., Introduction to X-Ray Powder Diffractometry, John Wiley & Sons, New York (1996); Bunn, ed., Chemical Crystallography, Clarendon Press, London (1948); and Klug & Alexander eds., X-ray Diffraction Procedures, John Wiley & Sons, New York (1974)) and were obtained as described below:

A Bragg-Brentano parafocusing powder X-ray diffractometer using monochromatic CuKαradiation (45 kV and 40 mA) was used for the analyses. The primary optics contained soller slits and an automatic divergence slit. Flat samples were prepared on zero background plates that were rotated during the measurements. The secondary optics contained soller slits, an automatic anti scatter slit, a receiving slit and a monochromator. The diffracted signal was detected with a proportional xenon-filled detector. Diffraction patterns were collected between 2°≦2θ (theta)≦40° in a continuous scan mode with a step size of 0.016° 2θ at a rate of 4° 2θ per minute. Raw data were stored electronically. Evaluation was performed on raw or smoothed diffraction patterns.

A Panalytical X'pert PRO MPD θ-θ diffractometer in reflection mode was used for the above-mentioned measurements. A person skilled in the art can set up instrumental parameters for a powder X-ray diffractometer so that diffraction data comparable to the data presented can be collected.

The nomenclature used for the compounds was generated using ACD/Name 8.00, Release Product Version 8.05.

The following abbreviations are used:

DMSO dimethyl sulfoxide;

### DMF N-dimethylformamide;

THF tetrahydrofuran;
TFA trifluoroacetic acid;
XRPD X-ray powder diffraction

### EXAMPLE 1

### N-{5-Chloro-2-[((2S)-3-{[1-(4-chlorobenzyl)piperidin-4-yl]amino}-2-hydroxy-2-methylpropyl)oxy]-4-hydroxyphenyl}propaneamide di-trifluoroacetate

### (i) N-(2-Hydroxy-4-methoxyphenyl)propanamide

To an ice-cooled solution of 2-hydroxy-4-methoxyaniline.HCl (600 mg, 3.4 mmol) and triethylamine (3 eq) in dichloromethane (25 mL) propionic anhydride (1.1 eq) was added dropwise. The solution was left at ambient temperature for 5 h. The reaction was quenched with water, the layers separated and the organic phase extracted with 1N NaOH (aq) (3×25 mL). The pH of the aqueous phase was adjusted with concentrated HCl to 5 and extracted with dichloromethane (3×25 mL). The organic phase was dried over anhydrous sodium sulphate, filtered and removed in vacuo, providing the subtitled compound as a brown solid (555 mg, 83%).

¹H NMR (300 MHz, CDCl₃— d₆) δ 7.04 (b), 6.83 (d, J=8.4, 1H), 6.58 (d, J=2.8, 1H), 6.43 (dd, J₁=8.4, J₂=2.8, 1H), 3.77 (s, 3H), 2.49 (q, J=7.6, 2H), 1.29 (t, J=7.5, 3H); APCI-MS: m/z 196 [MH⁺].

### (ii) N-(5-Chloro-2-hydroxy-4-methoxyphenyl)propanamide

To an ice-cooled solution of N-(2-hydroxy-4-methoxyphenyl)propanamide (500 mg, 2.6 mmol) and dimethylformamide hydrogen chloride (1 eq) in DMF (5 mL), MCPBA (70%, 1 eq) was added in small portions. The reaction was stirred for an additional 5 minutes, afterwhich it was quenched with 1M NaHCO₃ (aq) (50 mL). The aqueous phase was washed with ethyl acetate (50 mL). The organic phase was washed with water (3×25 mL), dried and removed in vacuo, providing the subtitled compound as a purple solid (408 mg, 71%).

¹HNMR (300 MHz, acetone-d₆) δ 9.68 (b, 1H), 9.12 (b, 1H), 7.37 (s, 1H), 6.62 (s, 1H), 3.83 (s, 3H), 2.49 (q, J=7.7, 2H), 1.18 (t, J=7.5, 3H); APCI-MS: m/z 229 [M⁺].

### (iii) N-(5-Chloro-4-methoxy-2-{[(2S)-methyloxiran-2-yl]methoxy}phenyl)propanamide

A suspension of N-(5-Chloro-2-hydroxy-4-methoxyphenyl)propanamide (202 mg, 0.88 mmol), [(2S)-2-methyloxiran-2-yl]methyl 3-nitrobenzenesulfonate (1 eq) and cesium carbonate (1.2 eq) in DMF (4 mL) was stirred at room temperature for 4 h. The mixture was separated over water (50 mL) and ethyl acetate (50 mL). The organic phase was washed with water (2×30 mL), dried and removed in vacuo, providing the subtitled compound as an off-white solid (249 mg, 95%).

¹HNMR (300 MHz, CDCl₃) δ 8.43 (s, 1H), 7.80 (b, 1H), 6.61 (s, 1H), 4.14 (m, 1H), 3.98 (m, 1H), 3.85 (s, 3H), 2.94 (m, 1H), 2.79 (m, 1H), 2.42 (q, J=7.6, 2H), 1.47 (s, 3H), 1.25 (t, J=7.5, 3H); APCI-MS: m/z 299 [MH⁺].

### (iv) N-{5-Chloro-2-[((2S)-3-{[1-(4-chlorobenzyl)piperidin-4-yl]amino}-2-hydroxy-2-methylpropyl)oxy]-4-hydroxyphenyl}propaneamide di-trifluoroacetate

To a solution of 1-(4-chlorobenzyl)-piperidin-4-yl amine (50 mg, 0.2 mmol) and N-(5-chloro-4-methoxy-2-{[(2S)-methyloxiran-2-yl]methoxy}phenyl)propanamide (1 eq) in acetonitrile (5 mL), lithium perchlorate (10 eq) was added. The reaction mixture was refluxed for 18 h. The reaction mixture was poured over a MEGA BE-SCX column (Bond Elut®, 5 g, 20 mL). The column was first washed with methanol (3×10 mL) and subsequently with a mixture of ammonia/methanol (1/20, 3×10 mL). The basic layers were pooled and the solvent removed in vacuo, providing N-{5-chloro-2-[((2S)-3-{[1-(4-chlorobenzyl)piperidin-4-yl]amino}-2-hydroxypropyl)oxy]-4-methoxyphenyl}propaneamide as a light brown oil (100 mg, 86%), which was redissolved in dichloromethane (4 mL). The solution was cooled to 0° C. and 1 M BBr₃ in dichloromethane (1 mL) added dropwise. The reaction was stirred for 18 h, after which it was quenched with methanol. The solvent was removed in vacuo and the residue purified by reverse phase prep. HPLC, using acetonitrile and water containing 0.1% TFA in gradient as mobile phase. Pooled fractions were freeze-dried to give the titled product as an amorphous white solid (38 mg, 39%).

¹H NMR (300 MHz, acetone-d₆) δ 8.66 (broad), 8.09 (3, 1H), 7.60 (d, J=8.4, 4H), 7.47 (d, J=8.4, 4H), 6.78 (s, 1H), 4.41 (s, 2H), 4.10-3.93 (m, 2H), 3.70-3.65 (m, 4H), 3.44-2.39 (m, 1H), 3.20 (m, 2H), 2.52-2.37 (m, 6H), 1.38 (s, 3H), 1.10 (t, J=7.5, 3H); APCI-MS: m/z 510 [MH⁺].

### EXAMPLE 2

### N-{5-Chloro-2-[((2S)-3-{[1-(4-chlorobenzyl)piperidin-4-yl]amino}-2-hydroxy-2-methylpropyl)oxy]-4-hydroxyphenyl}acetamide di-trifluoroacetate

Prepared according to the method described in example 1(i) reacting 2-hydroxy-4-methoxyaniline.HCl with acetic anhydride (1.1 eq).

¹H NMR (300 MHz, acetone-d₆) δ 8.77 (s, 1H), 8.06 (s, 1H), 7.61 (d, J=8.2 Hz, 2H), 7.47 (d, J=8.6 Hz, 2H), 6.79 (s, 1H), 4.43 (s, 2H), 4.08 (d, J=9.9 Hz, 1H), 3.94 (d, J=9.9 Hz, 1H), 3.79-3.61 (m, 3H), 3.68 (d, J=12.5 Hz, 1H), 3.42 (d, J=12.7 Hz, 1H), 3.32-3.13 (m, 2H), 2.63-2.48 (m, 2H), 2.49-2.29 (m, 2H), 2.08 (s, 3H), 1.38 (s, 3H); APCI-MS: m/z 496 [MH⁺].

### EXAMPLE 3

### N-{5-Chloro-2-[((2S)-3-{[1-(4-chlorobenzyl)piperidin-4-yl]amino}-2-hydroxy-2-methylpropyl)oxy]-4-hydroxyphenyl}acetamide

N-{5-chloro-2-[((2S)-3-{[1-(4-chlorobenzyl)piperidin-4-yl]amino}-2-hydroxy-2-methylpropyl)oxy]-4-hydroxyphenyl}acetamide di-trifluoroacetate (2.77 g) and ammonium hydroxide, 28% solution in water (2 eq) are dissolved in acetonitrile (20 mL). The mixture is extracted between water (100 mL) and dichloromethane (150 mL). The organic phase is washed three times with water (100 mL), dried and removed in vacuo yielding N-{5-chloro-2-[((2S)-3-{[1-(4-chlorobenzyl)piperidin-4-yl]amino}-2-hydroxy-2-methylpropyl)oxy]-4-hydroxyphenyl}acetamide (1.56 g, 63%) as the free base.

¹H NMR (300 MHz, acetone-d₆) δ8.48 (s, 1H), 8.15 (s, 1H), 7.32 (s, 4H), 6.71 (s, 1H), 3.89 (m, 2H), 3.59 (s, 2H), 3.43 (s, 2H), 2.83-2.67 (m, 4H), 2.45-2.40 (m, 1H), 2.07 (s, 3H), 2.03-1.97 (m, 2H), 1.85-1.82 (m, 2H), 1.39-1.19 (m, 2H), 1.25 (s, 3H); APCI-MS: m/z 496 [MH⁺].

### EXAMPLE 4

### N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph B)

A solution of N-{5-chloro-2-[((2S)-3-{[1-(4-chlorobenzyl)piperidin-4-yl]amino}-2-hydroxy-2-methylpropyl)oxy]-4-hydroxyphenyl}acetamide (50 mg) in acetonitrile (1 mL) is warmed to 60° C. A solution of benzoic acid (2 eq) in acetonitrile (1 mL; 40-70° C.) is added. The mixture is cooled to 30° C. and seeded with acetamide, N-[2-[(2S)-3-[[1-[(4-chlorophenyl)methyl]-4-piperidinyl]amino]-2-hydroxy-2-methylpropoxy]-4-hydroxyphenyl]-, benzoate salt (and the precipitate (polymorph B) collected (30 mg, 48%) and than allowed to cool to room temperature.

¹H NMR (300 MHz, DMSO-d₆) δ 9.02 (s, 1H), 7.94-7.92 (m, 2H), 7.76 (s, 1H), 7.57-7.54 (m, 2H), 7.47-7.43 (m, 2H), 7.37-7.35 (m, 2H), 7.30-7.27 (m, 2H), 6.63 (s, 1H), 3.80-3.74 (m, 2H), 3.40 (s, 2H), 2.80-2.72 (m, 4H), 2.00 (s, 3H), 1.95-1.89 (m, 2H), 1.82-1.80 (m, 2H), 1.34-1.31 (m, 2H), 1.20 (s, 3H); APCI-MS: m/z 496 [MH⁺]; mp (uncorrected) 111° C.

Polymorph B exhibits at least the characteristic X-ray powder diffraction (XRPD) peaks shown in FIG. 2, (expressed in degrees 2θ) (the margin of error being consistent with the United States Pharmacopeia, 25th ed. Rockville, Md.: United States Pharmacopeial Convention; 2002:2088-2089).

### EXAMPLE 5

### N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph A)

N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate (24 mg, polymorph B, example 4) is suspended in a mixture of ethylacetate (4.2 mL) and cyclohexane (7.8 mL). From this mixture, 1 ml is taken and the solvent is evaporated at 40° C., after which the solid (polymorph A) is collected and characterized.

mp (uncorrected) 107° C.

Polymorph A exhibits at least the characteristic X-ray powder diffraction (XRPD) peaks shown in FIG. 1, (expressed in degrees 2θ) (the margin of error being consistent with the United States Pharmacopeia, 25th ed. Rockville, Md.: United States Pharmacopeial Convention; 2002:2088-2089).

### EXAMPLE 6

### N-{5-Chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph A)

A solution of N-{5-chloro-2-[((2S)-3-{[1-(4-chlorobenzyl)piperidin-4-yl]amino}-2-hydroxy-2-methylpropyl)oxy]-4-hydroxyphenyl}acetamide (50 mg) in acetonitrile (1 mL) is warmed to 60° C. A solution of 2-furoic acid (2 eq) in acetonitrile (1 mL; 40-70° C.) is added. The mixture is left to cool down to room temperature and the precipitate (polymorph A) collected (49 mg, 78%).

¹H NMR (300 MHz, DMSO-d₆) δ 9.04 (s, 1H), 7.76 (s, 1H), 7.74 (s, 1H), 7.38-7.28 (m, 4H), 6.96 (m, 1H), 6.64 (s, 1H), 6.54 (m, 1H), 3.81-3.76 (m, 2H), 3.42 (s, 2H), 2.94-2.63 (m, 5H), 2.01 (s, 3H), 1.96-1.86 (m, 4H), 1.43-1.41 (m, 2H), 1.23 (s, 3H); APCI-MS: m/z 496 [MH⁺]; mp (uncorrected) 143° C.

Polymorph A exhibits at least the characteristic X-ray powder diffraction (XRPD) peaks shown in FIG. 3, (expressed in degrees 2θ) (the margin of error being consistent with the United States Pharmacopeia, 25th ed. Rockville, Md.: United States Pharmacopeial Convention; 2002:2088-2089).

### EXAMPLE 7

### N-{5-Chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph B

To a solution of N-{5-chloro-2-[((2S)-3-{[1-(4-chlorobenzyl)piperidin-4-yl]amino}-2-hydroxy-2-methylpropyl)oxy]-4-hydroxyphenyl}acetamide (2.44 g) in 2-butanol (50 mL) is added a solution of 2-furoic acid (2 eq) in 2-butanol (25 mL) is added. The mixture is slurred for 15 h and the precipitate (polymorph B) collected (2.32 g, 78%).

¹H NMR (300 MHz, DMSO-d₆) δ 9.04 (s, 1H), 7.76 (s, 1H), 7.74 (s, 1H), 7.38-7.28 (m, 4H), 6.96 (m, 1H), 6.64 (s, 1H), 6.54 (m, 1H), 3.81-3.76 (m, 2H), 3.42 (s, 2H), 2.94-2.63 (m, 5H), 2.01 (s, 3H), 1.96-1.86 (m, 4H), 1.43-1.41 (m, 2H), 1.23 (s, 3H); APCI-MS: m/z 496 [MH⁺]; mp (uncorrected) 167° C.

Polymorph B exhibits at least the characteristic X-ray powder diffraction (XRPD) peaks shown in FIG. 4, (expressed in degrees 2θ) (the margin of error being consistent with the United States Pharmacopeia, 25th ed. Rockville, Md.: United States Pharmacopeial Convention; 2002:2088-2089).

### EXAMPLE 8

### N-{5-Chloro-2-[((2S)-3-{[1-(4-chlorobenzyl)piperidin-4-yl]amino}-2-hydroxypropyl)oxy]-4-hydroxyphenyl}acetamide di-trifluoroacetate

Prepared according to the method described in example 2(iii) reacting N-(5-chloro-2-hydroxy-4-methoxyphenyl)acetamide with S-(+)-glycidyl nosylate (1 eq).

¹H NMR (300 MHz, acetone-d₆) δ 8.64 (broad, NH), 8.21 (s, 1H), 7.59 (d, J=9.0 Hz, 2H), 7.47 (d, J=9.0 Hz, 2H), 6.74 (s, 1H), 4.41-4.35 (m, 3H), 4.13-4.01 (m, 2H), 3.69-3.40 (m, 5H), 3.14 (m, 2H), 2.55-2.47 (m, 2H), 2.31 (m, 2H), 2.09 (s, 3H); APCI-MS: m/z 482 [MH⁺].

### EXAMPLE 9

### N-{5-Chloro-2-[((2S)-3-{[1-(4-chlorobenzyl)piperidin-4-yl]amino}-2-hydroxylpropyl)oxy]-4-hydroxyphenyl}propaneamide di-trifluoroacetate

Prepared according to the method described in example 2(iii) reacting N-(5-chloro-2-hydroxy-4-methoxyphenyl)acetamide with S-(+)-glycidyl nosylate (1 eq).

¹H NMR (300 MHz, DMSO-d₆) δ 10.05 (broad), 9.78 (broad), 9.79 (broad), 9.00 (broad), 8.88 (broad), 7.79 (m, 1H), 7.62-7.50 (m, 4H), 6.63 (s, 1H), 5.98 (broad), 4.29 (m, 2H), 4.16 (m, 1H), 3.95-3.88 (m, 2H), 341-2.97 (m, 7H), 2.35-2.22 (m, 4H), 1.82-1.75 (m, 2H), 1.07 (m, 3H); APCI-MS: m/z 496 [MH⁺].

### Human CCR1 Binding Assay

### Membranes

HEK293 cells, from ECACC, stably expressing recombinant human CCR1 (HEK-CCR1) were used to prepare cell membranes containing CCR1. The membranes were stored at −70° C. The concentration of membranes of each batch was adjusted to 10% specific binding of 33 pM [¹²⁵I] MIP-1α.

### Binding Assay

100 μL of HEK-CCR1 membranes diluted in assay buffer pH 7.4 (137 mM NaCl (Merck, Cat No 1.06404), 5.7 mM Glucose (Sigma, Cat No G5400), 2.7 mM KCl (Sigma, Cat No P-9333), 0.36 mM NaH₂PO₄×H₂O (Merck, Cat No 1.06346), 10 mM HEPES (Sigma, Cat No H3375), 0.1% (w/v) Gelatine (Sigma, Cat No G2625)) with the addition of 17500 units/L Bacitracin (Sigma, Cat No B 1025) were added to each well of the 96 well filter plate (0.45 μm opaque Millipore cat no MHVB N4550). 12 μL of compound in assay buffer, containing 10% DMSO, was added to give final compound concentrations of 1×10^{-5.5}-1×10^{-9.5} M. 12 μl cold human recombinant MIP-1α (270-LD-050, R&D Systems, Oxford, UK), 10 nM final concentration in assay buffer supplemented with 10% DMSO, was included in certain wells (without compound) as non-specific binding control (NSB). 12 μl assay buffer with 10% DMSO was added to certain wells (without compound) to detect maximal binding (B0).

12 μL [¹²⁵I] MIP-1α, diluted in assay buffer to a final concentration in the wells of 33 pM, was added to all wells. The plates with lid were then incubated for 1.5 hrs at room temperature. After incubation the wells were emptied by vacuum filtration (MultiScreen Resist Vacuum Manifold system, Millipore) and washed once with 200 μl assay buffer. After the wash, all wells received an addition of 50 μL of scintillation fluid (OptiPhase “Supermix”, Wallac Oy, Turko, Finland). Bound [¹²⁵I] MIP-1α was measured using a Wallac Trilux 1450 MicroBeta counter. Window settings: Low 5-High 1020, 1-minute counting/well.

### Calculation of Percent Displacement and IC₅₀

The following equation was used to calculate percent displacement.

Percent displacement=1−((cpm test−cpm NSB)/(cpm B0−cpm NSB)) where:

cpm test=average cpm in duplicate wells with membranes and compound and [¹²⁵I] MIP-1α cpm;
NSB=average cpm in the wells with membranes and MIP-1α and [¹²⁵I] MIP-1α (non-specific binding) cpm;
B0=average cpm in wells with membranes and assay buffer and [¹²⁵I] MIP-1α (maximum binding).

The molar concentration of compound producing 50% displacement (IC₅₀) was derived using the Excel-based program XLfit (version 2.0.9) to fit data to a 4-parameter logistics function.

### ABBREVIATIONS

MIP Macrophage Inflammatory Protein

HEK Human Embryonic Kidney cells

ECACC European Collection of Cell Cultures

HEPES N-(2-Hydroxyethyl)piperazine-N′-(2-ethanesulfonic acid, sodium)

CCR1 Chemokine Receptor 1

TABLE 1
The results obtained for the example compounds according to the present
invention, together with corresponding comparative examples.

| Compound | | | IC₅₀ (nM) |
|---|---|---|---|
| Example 1 | | | 0.4 |
| 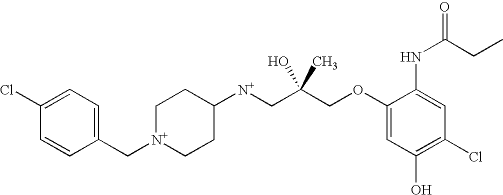 | | | |
| | 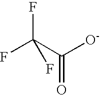 | 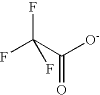 | |
| Comparison 1 | | | 7.0 |
| 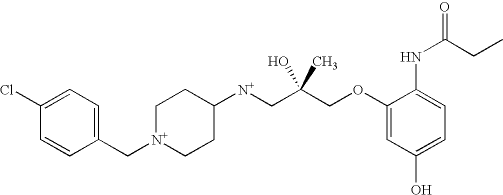 | | | |
| | 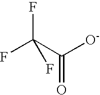 | 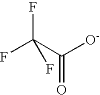 | |
| Example 2 | | | 0.7 |
| 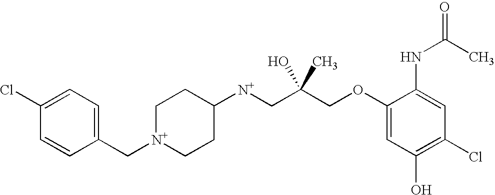 | | | |
| | 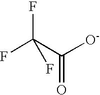 | 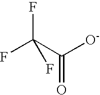 | |
| Comparison 2 | | | 3.9 |
| 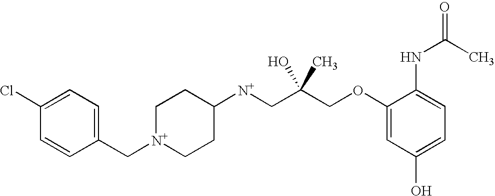 | | | |
| | 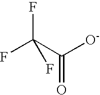 | 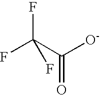 | |
| Example 8 | | | 1.6 |
| 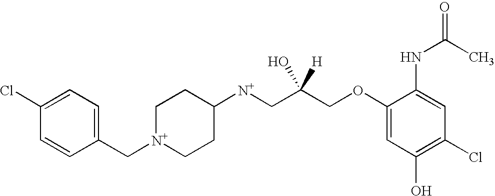 | | | |
| | 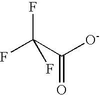 | 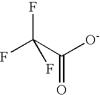 | |
| Comparison 8 | | | 4.5 |
| 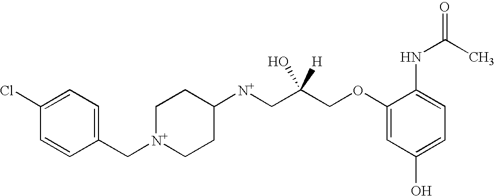 | | | |
| | 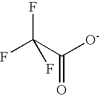 | 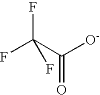 | |
| Example 9 | | | 2.7 |
| 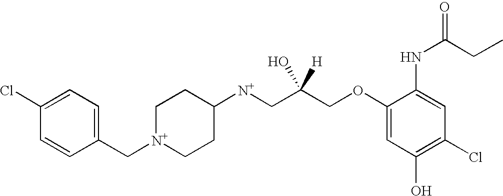 | | | |
| | 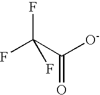 | 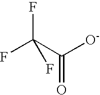 | |
| Comparison 9 | | | 13.6 |
| 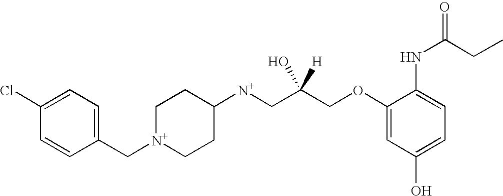 | | | |
| | 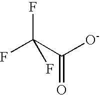 | 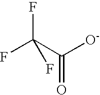 | |

1. A compound of general formula 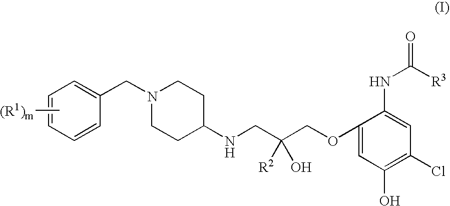
wherein
m is 1 or 2;
each R¹ independently represents halogen;
R² represents a hydrogen atom or a methyl group; and
R³ represents C₁-C₄ alkyl;
or a pharmaceutically acceptable salt or solvate thereof.

2. A compound according to claim 1, wherein m is 1 and R¹ represents a chlorine atom.

3. A compound according to claim 2, wherein R¹ is a chorine in the 4-position of the benzene ring relative to the carbon atom to which the CH₂ linking group is attached.

4. A compound according to claim 1, wherein R² represents a methyl group.

5. A compound according to claim 1, wherein R³ represents methyl.

6. A compound according to claim 1, wherein R³ represents ethyl.

7. A compound according to claim 1, which is
N-{5-Chloro-2-[((2S)-3-{[1-(4-chlorobenzyl)piperidin-4-yl]amino}-2-hydroxypropyl)oxy]-4-hydroxyphenyl}acetamide,
N-{5-Chloro-2-[((2S)-3-{[1-(4-chlorobenzyl)piperidin-4-yl]amino}-2-hydroxy-2-methylpropyl)oxy]-4-hydroxyphenyl}acetamide,
N-{5-Chloro-2-[((2S)-3-{[1-(4-chlorobenzyl)piperidin-4-yl]amino}-2-hydroxypropyl)oxy]-4-hydroxyphenyl}propaneamide, or
N-{5-Chloro-2-[((2S)-3-{[1-(4-chlorobenzyl)piperidin-4-yl]amino}-2-hydroxy-2-methylpropyl)oxy]-4-hydroxyphenyl}propaneamide;
or a pharmaceutically acceptable salt or solvate thereof.

8. N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate.

9. N-{5-Chloro-2-[((2S)-3-{[1-(4-chlorobenzyl)piperidin-4-yl]amino}-2-hydroxy-2-methylpropyl)oxy]-4-hydroxyphenyl}acetamide furoate.

10. N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph A), which exhibits X-ray powder diffraction peaks at d-values at 6.0, 16.3 and 19.1 Å.

11. N-{5-Chloro-2-[((2S)-3-{[1-(4-chlorobenzyl)piperidin-4-yl]amino}-2-hydroxy-2-methylpropyl)oxy]-4-hydroxyphenyl}acetamide benzoate (polymorph B), which exhibits X-ray powder diffraction peaks at d-values at 5.6, 10.4 and 14.3 Å.

12. N-{5-Chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph A), which exhibits X-ray powder diffraction peaks at d-values at 6.4, 16.8 and 18.1 Å.

13. N-{5-Chloro-2-[((2S)-3-{[1-(4-chlorobenzyl)piperidin-4-yl]amino}-2-hydroxy-2-methylpropyl)oxy]-4-hydroxyphenyl}acetamide furoate (polymorph B), which exhibits X-ray powder diffraction peaks at d-values at 6.7, 17.9 and 20.9 Å.

14. A process for preparing a compound of formula (I) or a pharmaceutically acceptable salt or solvate thereof as defined in claim 1 which comprises
(a) reacting a compound of formula 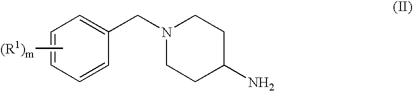
wherein m and R¹ are as defined in formula (I), with a compound of formula 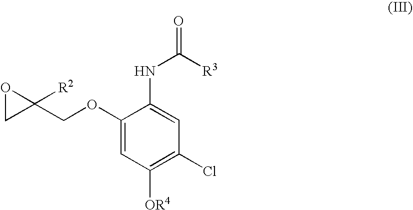
wherein R² and R³ are as defined in formula (I), and R⁴ represents a hydrogen atom or a suitable protecting group; or
(b) reacting a compound of formula 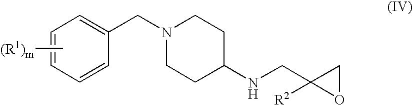
wherein m, R¹ and R² are as defined in formula (I), with a compound of formula 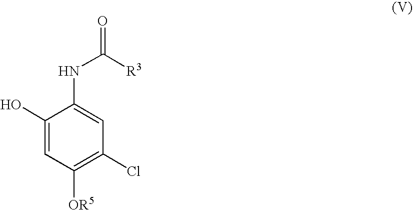
wherein R³ is as defined in formula (I), and R⁵ represents a hydrogen atom or a suitable protecting group; or
(c) reacting a compound of formula 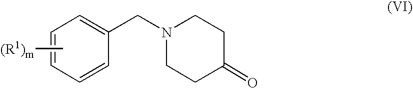
wherein m and R¹ are as defined in formula (I), with a compound of formula 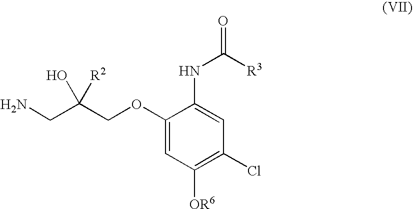
wherein R² and R³ are as defined in formula (I), and R⁶ represents a hydrogen atom or a suitable protecting group;
and optionally after (a), (b) or (c) forming a pharmaceutically acceptable salt or solvate of the compound of formula (I).

15. A pharmaceutical composition comprising a compound of formula (I), or a pharmaceutically acceptable salt or solvate thereof, as claimed in claim 1, in association with a pharmaceutically acceptable adjuvant, diluent or carrier.

16. A process for the preparation of a pharmaceutical composition which comprises mixing a compound of formula (I), or a pharmaceutically acceptable salt or solvate thereof, as claimed in claim 1, with a pharmaceutically acceptable adjuvant, diluent or carrier.

17-21. (canceled)

22. A method of treatment of inflammatory disease, respiratory disease and/or asthma, in a patient suffering from, or at risk of, said disease, which comprises administering to the patient a therapeutically effective amount of the compound of formula (I), or a pharmaceutically acceptable salt or solvate thereof, as claimed in claim 1.

23. An agent for the treatment of inflammatory disease, respiratory disease and/or asthma, which comprises as active ingredient a compound of formula (I), or a pharmaceutically acceptable salt or solvate thereof, as claimed in claim 1.

24. The method of claim 22, wherein the disease is a respiratory disease.

25. The method of claim 24, wherein the respiratory disease is a chronic respiratory disease.

26. The method of claim 22, wherein the disease is asthma.

27. A method for the treatment of a human disease or condition in which modulation of chemokine receptor 1 (CCR1) activity is beneficial, which comprises administering to a patient suffering from said disease or condition a therapeutically effective amount of a compound according to claim 1.
